(19) United States
(12) Patent Application Publication
(10) Pub. No.: US 2019/0091163 A1
(43) Pub. Date: Mar. 28, 2019

(54) NANO-SIZED DRUG DELIVERY STRUCTURE

(71) Applicants: INSTITUT NATIONAL DE LA SANTE ET DE LA RECHERCHE MEDICALE (INSERM), Paris (FR); UNIVERSITE D'AIX-MARSEILLE, MARSEILLE (FR); CENTRE NATIONAL DE LA RECHERCHE SCIENTIFIQUE(CNRS), PARIS CEDEX 16 (FR); INSTITUT JEAN PAOLI & IRENE CALMETTES, MARSEILLE (FR)

(72) Inventors: Richard CRASTER, London SW7 2AZ (GB); Tania PUVIRAJESINGHE, Marseille Cedex 09 (FR); Sébastien GUENNEAU, Marseille (FR); Zheng-liang ZHI, London WC2R 2LS (GB)

(21) Appl. No.: 16/096,941

(22) PCT Filed: Apr. 26, 2017
(86) PCT No.: PCT/EP2017/059966

§ 371 Date: Oct. 26, 2018

(30) Foreign Application Priority Data
Apr. 26, 2016 (EP) 16305485.1

(51) Int. Cl. A61K 9/51; A61K 38/04; A61K 47/64; A61K 47/69; A61K 31/00

(52) U.S. Cl. CPC A61K 9/5146; A61K 38/04; B82Y 5/00; A61K 47/6923; A61K 31/00; A61K 47/645

(57) ABSTRACT

The present invention relates to a novel multilayered assembly for the prolonged delayed and controlled diffusion of an active agent or a drug, in particular into an aqueous medium. In particular, the present invention relates to a nano-sized drug delivery structure comprising at least one support material; one multilayered structure overlaying and/or surrounding at least partially said support material and comprising at least two layers of oppositely charged materials; and one drug active agent conjugated by means of a disulphide bond on one or more negatively charged material(s) of one or more inner layer(s) of the said multilayered structure. Such a nano-sized drug delivery structure is more particularly useful for the manufacture of pharmaceutical compositions, for example intended to be used in cancer therapy. Internalization of the drug nanostructures into cancer cells, retardation of diffusion of the active drug, activity and specific targeting of the drug nanostructures were tested.

### FIELD OF THE INVENTION

The present application relates to a novel multilayered assembly for the prolonged delayed diffusion of an active agent or a drug in an aqueous medium.

More particularly, the present invention relates to a nano-sized drug delivery structure comprising a support material, a multilayered structure convenient for immobilizing and carrying at least an active agent or drug for its delayed and controlled diffusion in an aqueous medium, in particular at a specific target site.

### BACKGROUND OF THE INVENTION

Slow release formulas of drugs have important applications in tunable drug delivery.

A recurrent problem concerning pharmaceutical compositions is indeed that biological modification and/or elimination of the medication in the body leads to very short periods of effectiveness of the treatments. This is consequently compensated by the administration of repeated doses to obtain long term therapeutic levels of the considered drug.

Moreover, these drugs usually rapidly dissolve in the digestive tract and the total dosage is immediately fed into the blood of the treated individual. What is observed is then a high initial peak concentration followed by constantly decreasing concentration of the drug in the blood because of its biological elimination. Accordingly, there is little or no therapeutic effect at the end of the period between doses, which leads to a fluctuation of the therapeutic effect between doses corresponding to the peaks and valleys in the level of drug in the blood.

Many attempts have been made to develop timed-release pharmaceutical preparations which provide a more constant level of the drug in the blood over a prolonged period of time.

One common approach is to microencapsulate the medication, for example with a capsule wall material which provides a slower dissolution rate than free medication. Although the resultant blood level content is sustained, the medication is however still diffused into the body rapidly enough so there is an initially high blood level content which decreases quite rapidly within a few hours.

As a result, efforts have been made to adjust the rate of dissolution and, thus, control the timing of sustained drug release. For example, in U.S. No. 3,492,397, the dissolution rate is said to be controlled by adjusting the wax/ethyl cellulose ratio of an applied spray coating drug-coated pellets. However, when such microcapsules are administered, while the release of the active compound does not generally occur in the stomach, the coating is easily dissolved in the intestinal tract, thereby making the microcapsules porous. The porosity of the microcapsules thus promotes the rapid release of the active compound in the intestinal tract.

It appears that the sustained release drug delivery systems currently available are not completely satisfactory to the man skilled in the art.

Accordingly, a need exists for a structure allowing for the prolonged delayed diffusion of an active agent or a drug in an aqueous medium.

There is in particular a need for a structure allowing for the delayed and controlled diffusion of the drug or active agent in an aqueous medium.

There is also a need for a structure allowing for the delayed and controlled diffusion of a wide variety of drugs.

There is also a need for a delivery system providing constant blood concentration levels of a given drug or active agent over an extended period of time.

### SUMMARY OF THE INVENTION

The present invention aims to meet the here-above indicated needs.

The inventors unexpectedly identified that a particular nano-sized drug delivery structure comprising a support material, a multilayered structure and an active agent or drug allows for the delayed and controlled diffusion of the active agent or drug in an aqueous medium.

Accordingly, one of the objects of the present invention relates to a nano-sized drug delivery structure for delayed and controlled diffusion of at least one drug active, in particular into an aqueous medium, said drug delivery structure comprising at least:
one support material selected from the group consisting of:
a metal support material functionalized with at least one carboxylate-containing radical or at least one amine function,
a graphene-based or graphene derivative-based support material, and
a calcium carbonate nanoparticle support material;
one multilayered structure overlaying and/or surrounding at least partially said support material and comprising at least two layers of oppositely charged materials:
the positively charged material(s) being chosen from polylysine, chitin, chitosan, chitosan derivatives, amidated polymers, polyarginine, polyhistidine and mixtures thereof;
the negatively charged material(s) being selected from the group consisting of biopolymers, in particular from sulphated polysaccharides, more particularly selected from heparin, heparan sulphate, chrondroitin sulphate, dermatan sulphate, hyaluronic acid and polyglutamic acid; forms of carbon functionalized with at least one carboxylate-containing radical, in particular graphene oxide functionalized with at least one carboxylate-containing radical; and mixtures thereof; and
one drug active immobilized on one or more material(s) of one or more inner layer(s) of said multilayered structure.

More particularly, the present invention relates to a nano-sized drug delivery structure for delayed and controlled diffusion of at least one drug active, in particular into an aqueous medium, said drug delivery structure comprising at least:
one support material selected from the group consisting of:
a metal support material functionalized with at least one carboxylate-containing radical or at least one amine function,
a graphene-based or graphene derivative-based support material, and
a calcium carbonate or calcium phosphate nanoparticle support material;
one multilayered structure overlaying and/or surrounding at least partially said support material and comprising at least two layers of oppositely charged materials:
the positively charged material(s) being chosen from polylysine, chitin, chitosan, chitosan derivatives, amidated polymers, polyarginine, polyhistidine and mixtures thereof;
the negatively charged material(s) being selected from the group consisting of sulphated polysaccharides, more particularly selected from heparin, heparan sulphate, chrondroitin sulphate, dermatan sulphate, keratan sulphate, hyaluronic acid and polyglutamic acid; and
one drug active agent conjugated by means of a disulphide bond to one or more negatively charged material(s) of one or more inner layer(s) of the said multilayered structure.

Unexpectedly, the inventors discovered that the nano-sized drug delivery structure of the invention can be used for drug transportation and delivery. More particularly, they demonstrated that this nano-sized drug delivery structure consisting of a series of different components is capable of achieving controlled diffusion of the combined drug in an aqueous medium.

As disclosed here-after, they achieved a nano-sized drug layering delivery structure with an anisotropic homogeneous diffusivity. Independent research groups have applied a similar model to various applications implicating the protection of steel by using multilayered materials including concrete and sand to build a cloak against chloride ions penetration (Zeng, L. and R. Song, Sci Rep, 2013. 3: p. 3359) as well as for the design of water-based invisibility cloaks (Schittny, R., et al., 2014. 345(6195): p. 427-9). All these methods have been developed based on the well known Fick's laws of diffusion, whose expression in general transformed coordinates appears for the first time in (Guenneau, S. and T. M. Puvirajesinghe, J. Roy. Soc. Interface, 2013. 10, 20130106).

However, to the knowledge of the inventors, a nano-sized drug delivery structure according to the invention, in particular for providing a delayed and controlled diffusion of a drug active into an aqueous medium, has never been disclosed in the art.

In a preferred embodiment, the support material of the drug delivery structure of the invention is a graphene-based or a graphene derivative-based support material, in particular a graphene derivative-based support material, more particularly graphene oxide.

In a particular embodiment, the multilayered structure of the drug delivery structure of the invention comprises at least one layer of polylysine as positively charged material and at least one layer of heparin as negatively charged material.

In another particular embodiment, the multilayered structure of the drug delivery structure of the invention can comprise one or more layers of graphene oxide as negatively charged material.

In an embodiment, the drug active of the drug delivery structure of the invention is selected from the group consisting of peptides, proteins, small chemical compounds, and mixtures thereof, in particular peptides, more particularly cysteine-containing peptides, preferably cationic peptides, and even more particularly cationic lytic peptides.

Preferably, the drug active comprises at least one —SH radical available for immobilization, preferably conjugation, on a layer of the drug delivery structure of the invention, with the said immobilization through said —SH radical not affecting the activity of the drug active.

In particular, said drug active originally contains a sulfhydryl group or has been modified in order to contain a sulfhydryl group.

More preferably, the drug active comprises at least one cysteine available for immobilization, preferably conjugation, on a layer of the drug delivery structure of the invention, with the said immobilization through said cysteine not affecting the activity of the drug active.

A drug active of the invention comprising a —SH radical can for example be mertansine.

In an embodiment, the drug active of the drug delivery structure of the invention is selected from the group consisting of anticancer agents, antibacterial agents, antiviral agents, antifungal agents, analgesics, antihyperlipidemic agents, antidepressants, β-receptor blockers, calcium channel blockers, diuretics, cardiac glycosides, antiarrhythmics, nitrate, antianginals, vasoconstrictors, vasodilators, antihypertensive agents, agents affecting the central nervous system, agents for musculoskeletal disorders, antiallergy agents, mast cell inhibitors agents, anti-inflammatory agents, skin agents, eye disorders agents, obstetrics agents and gynecologic agents, and mixtures thereof, and is preferably at least one anticancer agent.

In a preferred embodiment, the multilayered structure of the invention comprises at least one inner layer of heparin onto which the at least one drug active is immobilized, preferably conjugated. In particular, this immobilization, more particularly conjugation, of the drug active(s) involves a disulphide bond.

In this embodiment, the multilayered structure of the drug delivery structure according to the invention preferably comprises at least one inner layer of heparin onto which the at least one drug active is conjugated by means of a disulphide bond.

In a particular embodiment, the support material is in the form of platelets or flakes. In particular, the support material is a graphene oxide in the form of platelets or flakes, more particularly is a graphene oxide in the form of aggregated or not aggregated platelets or flakes.

In this embodiment, the multilayered structure is preferably formed from at least one layer of polylysine as positively charged material and at least one layer of heparin as negatively charged material.

Preferably, the multilayered structure comprises at least one inner layer of heparin onto which the at least one drug active is immobilized, in particular conjugated, the immobilization, in particular conjugation, involving more particularly a disulphide bond.

In a particular embodiment, the drug delivery structure of the invention comprises at least:
(i) graphene oxide or graphene, preferably graphene oxide, as support material; and
(ii) a multilayered structure comprising at least:
   a first layer of positively charged polylysine;
   a second layer of negatively charged heparin onto which the at least one drug active is immobilized;
   a third layer of positively charged polylysine;
   a fourth layer of negatively charged heparin onto which the at least one drug active is optionally immobilized; and
   a fifth layer of positively charged polylysine.

Preferably, this multilayered structure further comprises:
an additional sixth layer of negatively charged heparin onto which the at least one drug active is optionally immobilized; and
an additional seventh layer of positively charged polylysine.

Optionally, the multilayered structure further comprises:
an additional eighth layer of negatively charged heparin onto which the at least one drug active is optionally immobilized; and
an additional ninth layer of positively charged polylysine.

The drug active immobilized on the different layers is independently selected, and can in particular be different or the same, more particularly the same.

In particular, when the support material of a drug delivery structure of the invention is in the form of platelets or flakes, both sides of the support material can, independently, be at least partially overlaid by the multilayered structure.

In a particular embodiment, the support material of a nano-sized drug delivery structure of the invention has a thickness of between 0.1 nm and 5 nm, preferably between 0.5 nm and 2 nm, in particular between 0.5 nm and 1 nm.

In a particular embodiment, the polylysine layer(s) of a multilayered structure of the invention has, independently, a thickness of between 1 nm and 10 nm.

In a particular embodiment, the heparin layer(s) of a multilayered structure of the invention has, independently, a thickness of between 1 nm and 10 nm.

According to a preferred embodiment of the invention, the multilayered structure of a nano-sized drug delivery structure of the invention is a 5-layered stack, a 7-layered stack or a 9-layered stack.

In another particular embodiment, the nano-sized drug delivery structure of the invention has a spherical or ellipsoidal architecture in the form of a core/shell type nanoparticle comprising a core formed in all or part of the support material and a shell formed of the multilayered structure surrounding at least partially the core.

Preferably, the support material of a nano-sized drug delivery structure according to this embodiment is formed, in whole or in parts, of gold nanoparticles functionalized with at least one carboxylate-containing radical.

In particular, the multilayered structure of a nano-sized drug delivery structure according to the invention is formed by alternating (i) layers of positively charged polylysine with (ii) layers of negatively charged heparin.

In a preferred embodiment, the nano-sized drug delivery structure according to the invention comprises, or even consists of:

(i) a support material formed of negatively charged graphene quantum dots or of a metal support material functionalized with at least one carboxylate-containing radical, preferably of negatively charged graphene quantum dots, in particular of carboxylated graphene quantum dots; and

(ii) a multilayered structure comprising, or consisting, of:
a first layer surrounding the said support material and formed of positively charged polylysine;
a second layer of negatively charged heparin onto which the at least one drug active is immobilized, in particular conjugated;
a third layer of positively charged polylysine;
a fourth layer of negatively charged heparin onto which the at least one drug active is optionally immobilized, in particular conjugated;
a fifth layer of positively charged polylysine;
a sixth layer of negatively charged heparin onto which the at least one drug active is optionally immobilized, in particular conjugated;
a seventh layer of positively charged polylysine, and optionally
a eighth layer of negatively charged heparin onto which the at least one drug active is optionally immobilized, in particular conjugated; and
a ninth layer of positively charged polylysine.

According to a preferred embodiment, the multilayered structure of a nano-sized drug delivery structure according to the invention consists in alternating layers of positively charged polylysine and layers of negatively charged heparin.

Advantageously, the multilayered structure of the drug delivery structure according to the invention is a 5-layered structure, a 7-layered structure or a 9-layered structure.

According to a particular embodiment, a targeting agent is immobilized, in particular conjugated, on the external surface of the outer layer of the multilayered structure of a nano-sized drug delivery structure according to the invention, the said outer layer being in particular a polylysine layer. Alternatively, said outer layer can be a heparin layer.

The targeting agent can preferably be selected from the group consisting of an antibody, an antibody fragment, an oligonucleotide, a peptide, an hormone, a ligand, a cytokine, a peptidomimetic, a protein, a carbohydrate, a chemically modified protein, a chemically modified nucleic acid, a chemically modified carbohydrate that targets a known cell-surface protein, and an aptamer, and is in particular an antibody or an antibody fragment, more particularly an antibody or an antibody fragment that targets cancer cells.

Another object of the present invention is a process for preparing a nano-sized drug delivery structure according to the invention, wherein the layers of the multilayered structure are formed by chemical deposition of each material in the appropriate order on the support material, the deposition steps being more particularly interspersed by washing, centrifugation and/or ultrafiltration steps.

According to an advantageous embodiment, the layers of the multilayered structure are formed by chemical deposition in an aqueous medium supplemented with a non-ionic surfactant, in particular a low-density non-ionic surfactant, said low-density non-ionic surfactant being preferably chosen from poloxamers.

A further object of the present invention is a use of a nano-sized drug delivery structure according to the invention for the manufacture of a pharmaceutical composition.

In particular, the drug active of the nano-sized drug delivery structure used is an anti-cancer agent, the said pharmaceutical composition being intended to be used in cancer therapy.

Another object of the present invention is a pharmaceutical composition comprising, in a pharmaceutically acceptable carrier, at least one nano-sized drug delivery structure according to the invention.

The present invention further relates to a medicament comprising at least one nano-sized drug delivery structure according to the invention.

### FIGURES' LEGENDS

FIG. 1: represents a nano-sized drug delivery structure according to a particular embodiment of the invention wherein:

(i) the support material is a graphene oxide in the form of a platelet or flake having a thickness of 1 nm, and

(ii) a multilayered structure overlaying the support material consisting in a first layer formed of positively charged polylysine as positive material; a second layer of negatively charged heparin onto which at least one drug active is conjugated; a third layer of positively charged polylysine; a fourth layer of negatively charged heparin; and a fifth layer of positively charged polylysine;

in which the polylysine layers have a thickness of 3.2 nm and the heparin layers have a thickness of 4.1 nm.

Antibodies are immobilized in particular by their constant part, onto the external face of the fifth layer, which is in the present case the outer layer.

FIG. 2: represents a cross-sectional drawing of a nano-sized drug delivery structure according to a particular embodiment of the invention wherein the nano-sized drug delivery structure has a spherical/ellipsoidal architecture in the form of a core/shell type nanoparticle, wherein:

(i) the core comprises a support material being gold nanoparticles and

(ii) a multilayered structure surrounding the support material consisting in a first layer formed of positively charged polylysine; a second layer of negatively charged heparin onto which at least one drug active is conjugated; a third layer of positively charged polylysine; a fourth layer of negatively charged heparin onto which at least one drug active is conjugated; a fifth layer of positively charged polylysine; a sixth layer of negatively charged heparin onto which at least one drug active is conjugated and a seventh layer of positively charged polylysine.

Antibodies are immobilized, in particular by their constant part, onto the external face of the seventh layer, which is in the present case the outer layer.

FIG. 3: schematically summarizes a procedure for the fabrication of the nano-sized drug delivery structure illustrated in the examples.

FIG. 4: represents the release of therapeutic anticancer drug upon hydrolysis of disulphide bond enabled in reducing conditions (MALDI spectroscopy analysis).

The top part illustrates the results obtained with the peptide linked to an FITC via an aminohexanoic acid (Ahx) spacer in H₂O heated for 1 hour at 56° C.

The following part illustrates the results obtained with the peptide linked to an FITC via an aminohexanoic acid (Ahx) spacer in the presence of a reducing agent Dithiothreitol (DTT) heated for 1 hour at 56° C.

The third part illustrates the results obtained with the peptide linked to an FITC via an aminohexanoic acid (Ahx) spacer, said peptide being immobilized on heparin, in the presence of a reducing agent (DTT) heated for 1 hour at 56° C.

The bottom part illustrates the results obtained with the peptide linked to an FITC via an aminohexanoic acid (Ahx) spacer, said peptide being immobilized on heparin, in H₂O heated for 1 hour at 56° C.

The same results have been obtained when heating 1 hour at 37° C.

Abscissa: compounds mass (m/z). Ordinate: relative intensity (in astronomical unit (a.u.))

FIG. 5: schematically represents the method used in order to determine the rate of diffusion of a drug active of the invention through a nano-sized drug delivery structure of the invention or any step of the preparation of said structure (for example through graphene oxide (GO) only).

FIG. 6: represents the retardation of diffusion using the metamaterial (nano-sized drug delivery structure) of the invention.

Are represented: the drug on the graphene oxide alone (GO); the drug on the couple graphene oxide with polylysine (GO-polylysine); a drug immobilized in a nano-sized drug delivery structure of the invention having a multilayered structure of 3 layers (3-layer); a drug immobilized in a nano-sized drug delivery structure of the invention having a multilayered structure of 5 layers (5-layer); a drug immobilized in a nano-sized drug delivery structure of the invention having a multilayered structure of 7 layers (7-layer); the drug alone (peptide).

Abscissa: time (hours). Ordinate: Fluorescence of the marked peptide (in relative fluorescence unit (RLU))

FIG. 7: represents a microscopic observation of the internalization of the drug delivery metamaterial of the invention into a breast cancer cell using transmission microscopy and fluorescence microscopy (peptide). The arrow shows the cellular internalization of the structure.

FIG. 8: represents the synthesis of heparin-mertansine from heparin-PDPH by displacement reaction at the disulphide linkage.

FIG. 9: represents the retardation of diffusion observed when using the nano-sized drug delivery structure of the invention.

Are represented: the drug on the graphene oxide alone (GO peptide control); a drug immobilized in a nano-sized drug delivery structure of the invention having a multilayered structure of 3 layers (3-layer); a drug immobilized in a nano-sized drug delivery structure of the invention having a multilayered structure of 5 layers (5-layer); a drug immobilized in a nano-sized drug delivery structure of the invention having a multilayered structure of 9 layers (9-layer); the drug alone (free peptide).

Abscissa: time (minutes). Ordinate: Peptide concentration (μM).

FIG. 10: represents the uptake efficiency of the GO nanodrug structures measured using various endocytosis inhibitors (Genistein, Cytochalasin D and Chlorpromazine) in comparison with the graphene oxide structure free of any drug active (GO control).

Abscissa: amount of endocytosis inhibitor (μM). Ordinate: uptake efficiency (%).

FIG. 11: represent the variation of uptake efficiency of GO-HER2 antibody nanodrug structures upon addition of Lapatinib.

Abscissa: amount of Lapatinib (μM). Ordinate: uptake efficiency (%).

FIG. 12: represents the localization of GO-HER2 nanoparticles in SKBR3 cells using transmission electron microscopy. A: Global localization in the cell. B: Specific localization in the lysosomal cell compartments using colocalization with the lysosomal marker, LAMP1.

FIG. 13: represents the retardation of diffusion observed when using the nano-sized drug delivery structure of the invention.

Are represented: the drug on the graphene oxide alone (GO); a drug immobilized in a nano-sized drug delivery structure of the invention having a multilayered structure of 3 layers (3-layer); a drug immobilized in a nano-sized drug delivery structure of the invention having a multilayered structure of 5 layers (5-layer) and all these nanoparticles free of drug (Ctrl GO).

A: Abscissa: nano-sized structure. Ordinate: Fluorescence of the marked peptide (in relative fluorescence unit (RLU)).

B: Abscissa: quantity of preparation (μL). Ordinate: Fluorescence of the marked peptide (in relative fluorescence unit (RLU)) in particular in a 9-layered structure.

FIG. 14: represents the release of mertansine drug from GO-HER2 nanodrug structures in four HER2-overexpressing breast cancer cell lines, SKBR3, CRL1500, CRL2331 and CRL2351.

Abscissa: nano-sized structure (9-layered GO-drug and Control). Ordinate: Cell proliferation (%).

FIG. 15: represents a drug penetration experiment using 3D cultured tumor spheroids.

Are represented: a drug immobilized in a nano-sized drug delivery structure of the invention (GO) (9-layered structure) and a nano-sized structure free of drug (Ctrl) in HER2 overexpressing cell line BT474 compared to lower HER2 expressing cell line, MDA-MB-231.

Abscissa: nano-sized structure and quantity of preparation (μL). Ordinate: reduction in cell proliferation (in relative fluorescence unit (RLU)).

### DETAILED DESCRIPTION OF THE INVENTION

### Definitions

As used herein:
“anisotropy” is described as the property of being directionally dependent, as opposed to isotropy which implies identical properties in all directions.
“X-layered stack” concerning a multilayered structure of a nano-sized drug delivery structure of the invention means that the multilayered structure comprises X layers of oppositely charged materials, more particularly of opposed and alternating charged material, i.e. if the first layer is positive, then the following layer is negative, and the further following layer is positive, etc.
“labelled” nano-sized drug support is intended for a support bearing a detectable molecule or substance and which can be distinguished from a support with no detectable molecule or substance, or a different detectable molecule or substance, using an imaging technique.
“pharmaceutically acceptable carrier” refers to a carrier medium which does not interfere with the effectiveness of the biological activity of the active ingredient(s) and which is not excessively toxic to the host at the concentration at which it is administered. The use of such media for pharmaceutically active substances is well known in the art (see for example “Remington's Pharmaceutical Sciences”, E. W. Martin, 18th Ed., 1990, Mack Publishing Co.: Easton, Pa.).
“individual” is intended for an animal, including human beings, affected or likely to be affected with a virus infection according to the invention. Said animal can in particular be intended for livestock, such as cattle, pigs and poultry; other non-human mammals such as pet, zoo or sports animals; or human beings. Said individual is preferably a human being.
A first layer of a multilayered structure according to the invention is the layer directly in contact with the support material. Considering that a multilayered structure of the invention has at least 2 layers, the first layer is obligatorily an inner layer.
An inner layer of a multilayered structure according to the invention is a layer at least partially overlaid with and/or surrounded by another layer according to the invention.
An outer layer of a multilayered structure according to the invention is a layer which is not at least partially overlaid with or surrounded by another layer according to the invention.
An aqueous medium according to the invention can be selected from the group consisting of water and aqueous saline mediums, and is preferably blood.

Support Material

A nano-sized drug delivery structure firstly comprises a support material. The rest of the drug delivery structure is build onto, over and/or around this support material.

A support material according to the invention is more particularly selected from the group consisting of:
a metal support material functionalized with at least one carboxylate-containing radical or with at least one amine function,
a graphene-based or graphene derivative-based support material, and
a calcium carbonate or calcium phosphate nanoparticle support material.

In a particular embodiment, the support material of a drug delivery structure of the invention is a metal support material functionalized with at least one carboxylate-containing radical.

A metal support material functionalized with at least one carboxylate-containing radical according to the invention can be selected from the group consisting of titanium functionalized with at least one carboxylate-containing radical, platinum functionalized with at least one carboxylate-containing radical, nickel functionalized with at least one carboxylate-containing radical, palladium functionalized with at least one carboxylate-containing radical, silver functionalized with at least one carboxylate-containing radical, gold functionalized with at least one carboxylate-containing radical and their derivatives.

As metal support material functionalized with at least one carboxylate-containing radical can preferably be selected from titanium functionalized with at least one carboxylate-containing radical, and can more preferably be a titanium oxide functionalized with at least one carboxylate-containing radical.

A metal, and in particular a metal as indicated here-above, can be functionalized with at least one carboxylate-containing radical by methods well known to the man skilled in the art such as Surface Modification and functionalization of metal and metal oxide nanoparticles by organic ligands, as illustrated in the review of Marie-Alexandra Neouze and Ulirich Schubert, Monatch Chem, 139, 183-195, 2008.

In another embodiment, the support material of a drug delivery structure of the invention is a metal support material functionalized with at least one amine function, preferably selected from amine-functionalized titanium, amine-functionalized platinum, amine-functionalized nickel, amine-functionalized palladium, amine-functionalized silver, amine-functionalized gold and their derivatives thereof.

An amine function of the invention can be a primary amine, a secondary amine or a tertiary amine.

In another embodiment, the support material of a drug delivery structure of the invention is a graphene-based or graphene derivative-based support material.

A graphene derivative-based support material can in particular be selected from the group consisting of graphene oxide, hydrogenated graphene, aminated-graphene, and aminated graphene oxide.

In particular, a support material according to the invention is selected from graphene, aminated graphene, graphene oxide and aminated graphene oxide, and is more preferably graphene oxide.

In another embodiment, the support material of a drug delivery structure of the invention is a calcium carbonate or calcium phosphate nanoparticle support material.

According to a particular embodiment, a support material of the invention is positively charged, and is in particular selected from amine-functionalized support materials, more particularly as defined here-above.

According to a particular embodiment, a support material of the invention is selected from a metal support material functionalized with at least one carboxylate-containing radical, a graphene-based or graphene derivative-based support material and an amine-functionalized metal support material, more particularly selected from a graphene-based or graphene derivative-based support material and an amine-functionalized metal support material, preferably selected from a graphene-based or graphene derivative-based support material, and more preferably selected from graphene, graphene oxide, an aminated graphene and an aminated graphene oxide.

In a particular embodiment, the support material is in the form of platelets or flakes.

According to this particular embodiment, the support material has a thickness comprised between 0.1 nm and 5 nm, preferably between 0.5 nm and 2 nm, in particular between 0.5 nm and 1 nm.

According to a particular embodiment, the support material of the invention in the form of platelets of flakes has a surface area between 25 nm² and 40,000 nm², in particular between 5,000 nm² and 25,000 nm², preferably between 8,000 nm² and 15,000 nm².

Preferably, the support material is a graphene oxide in the form of aggregated or not aggregated graphene oxide platelets or flakes, in particular aggregated or not aggregated nanosized graphene oxide platelets or flakes.

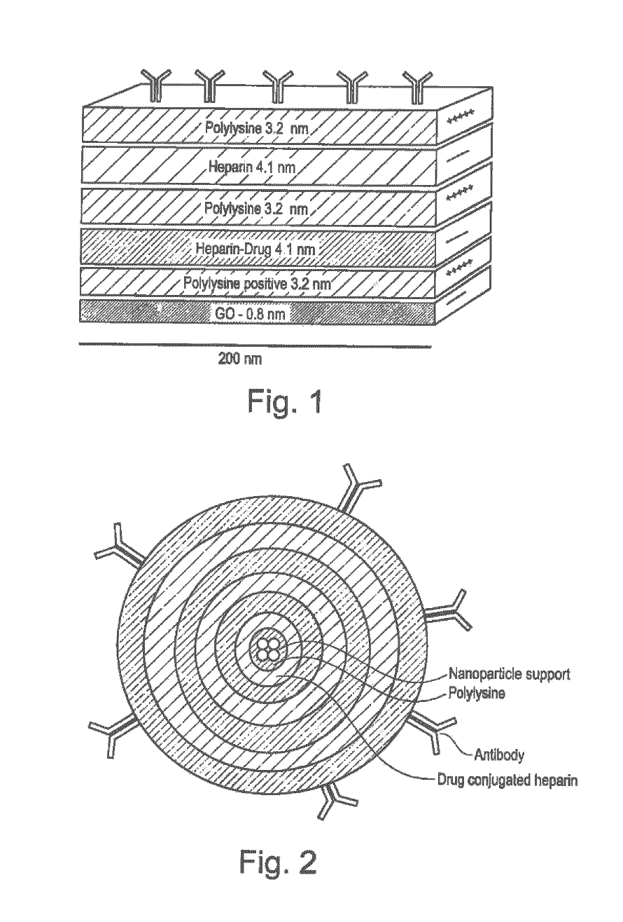

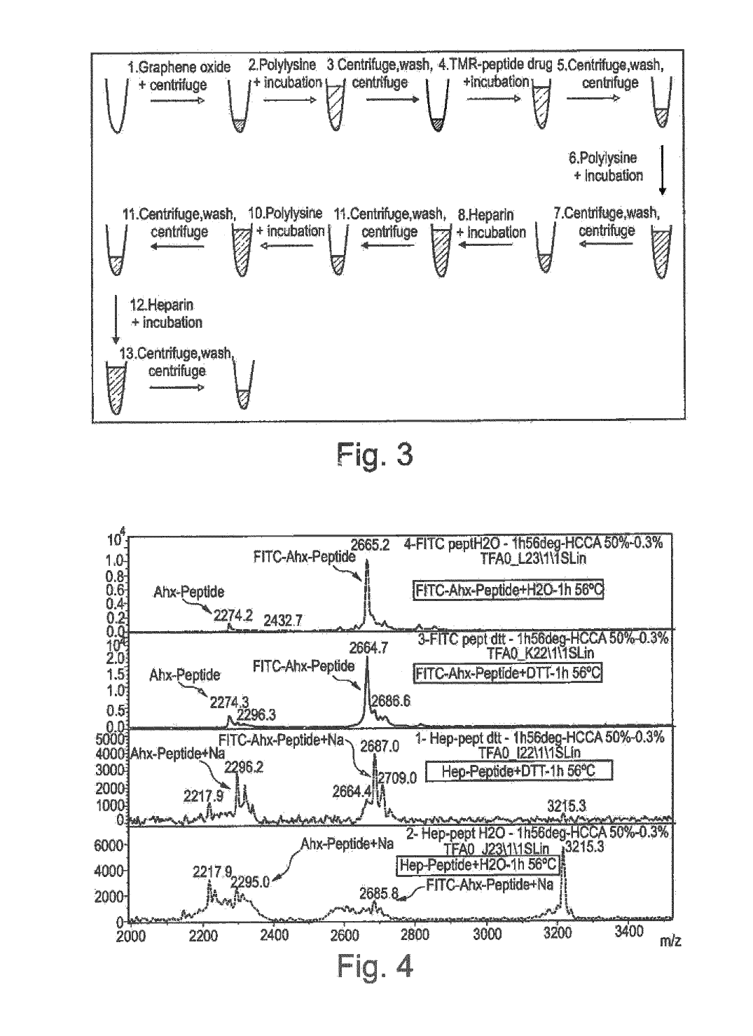

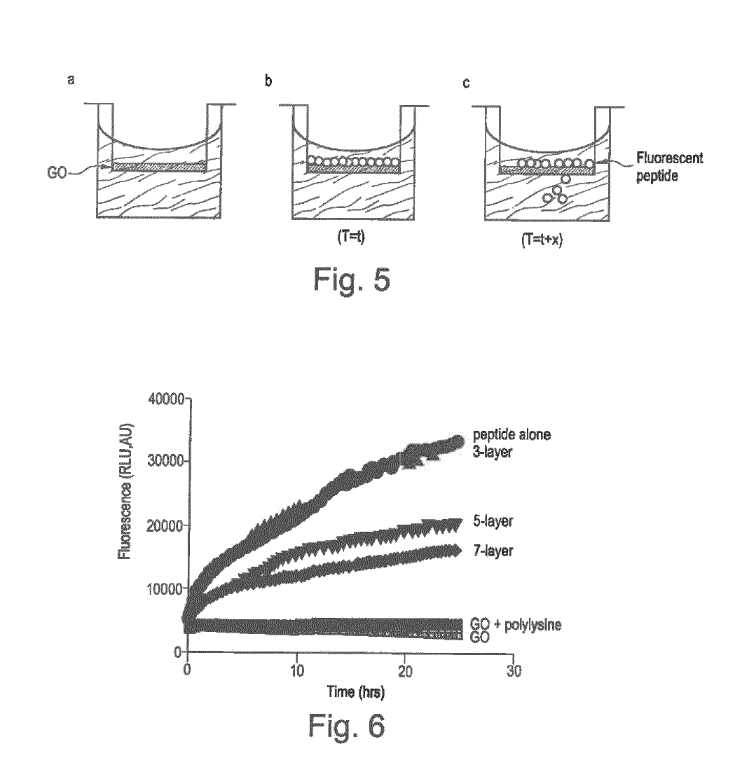

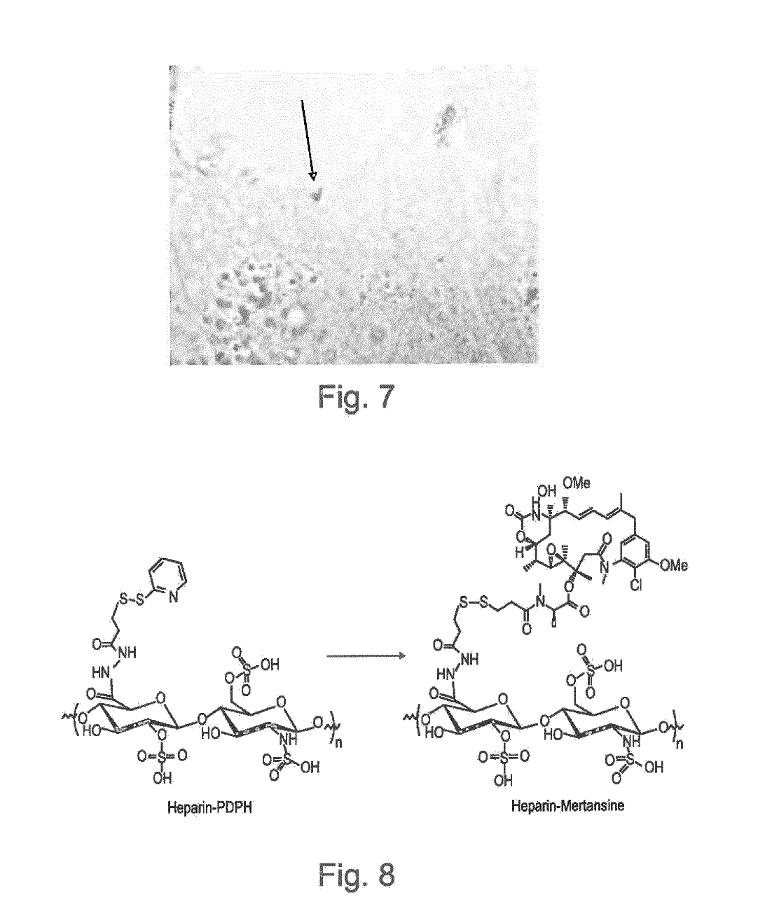

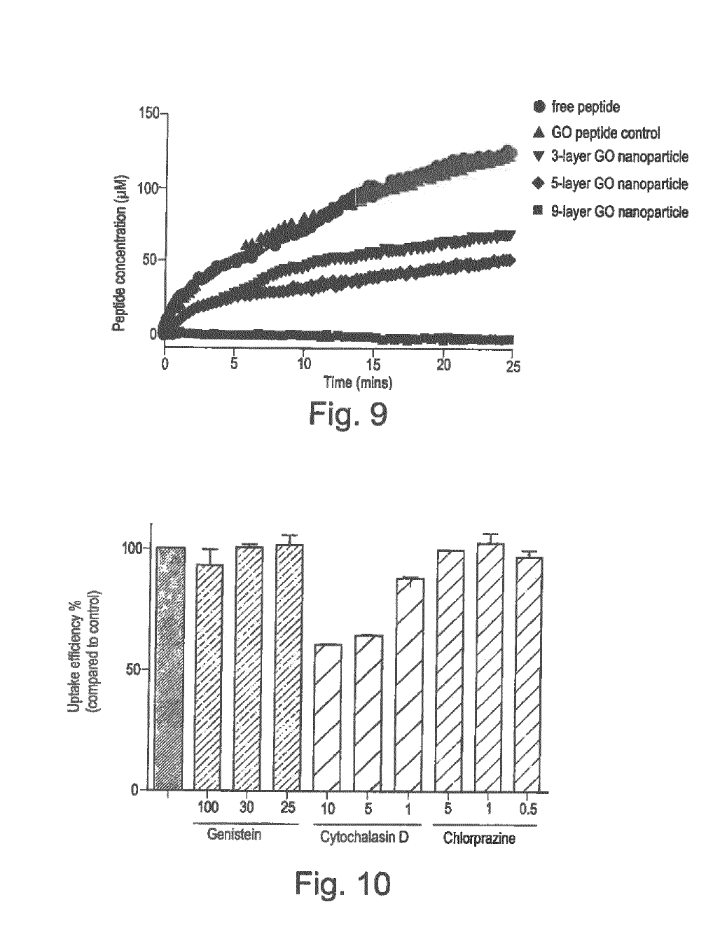

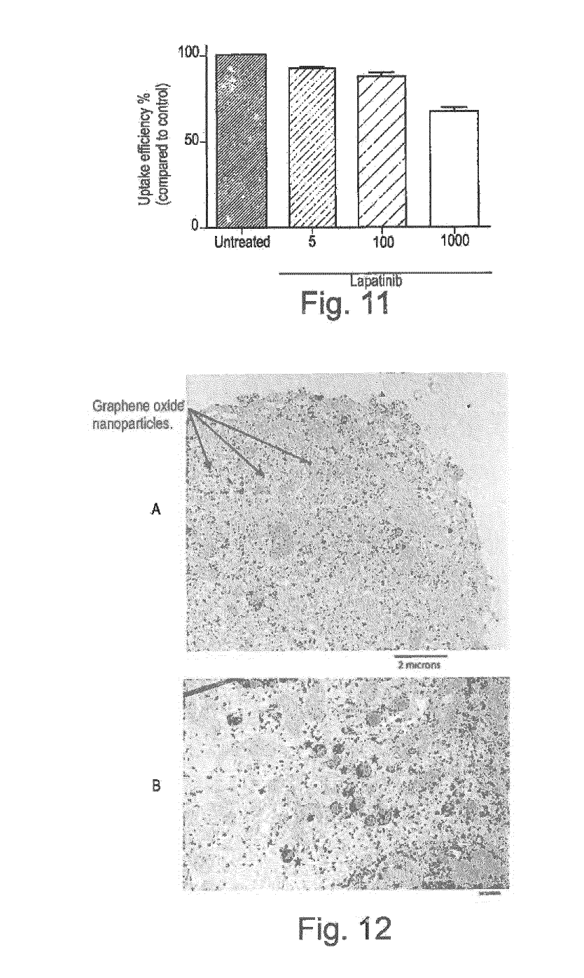

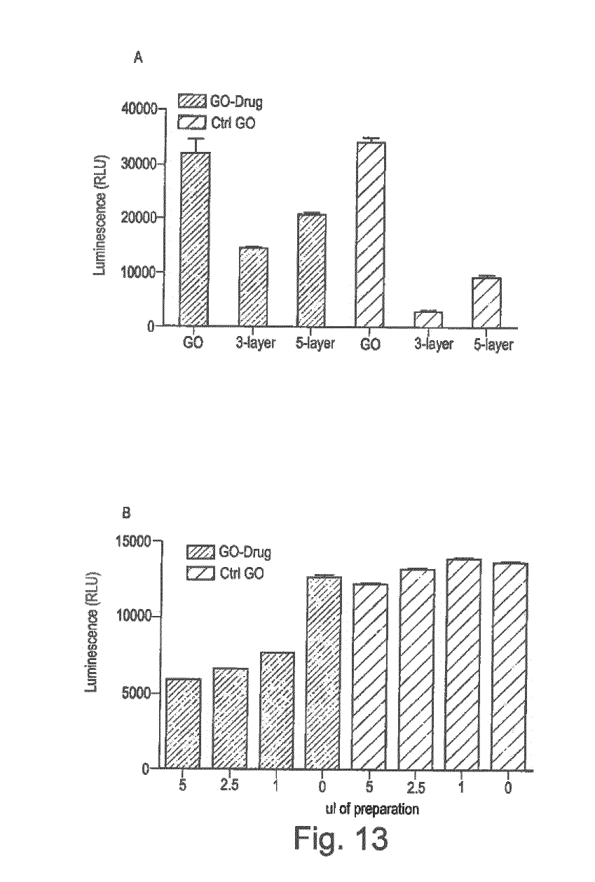

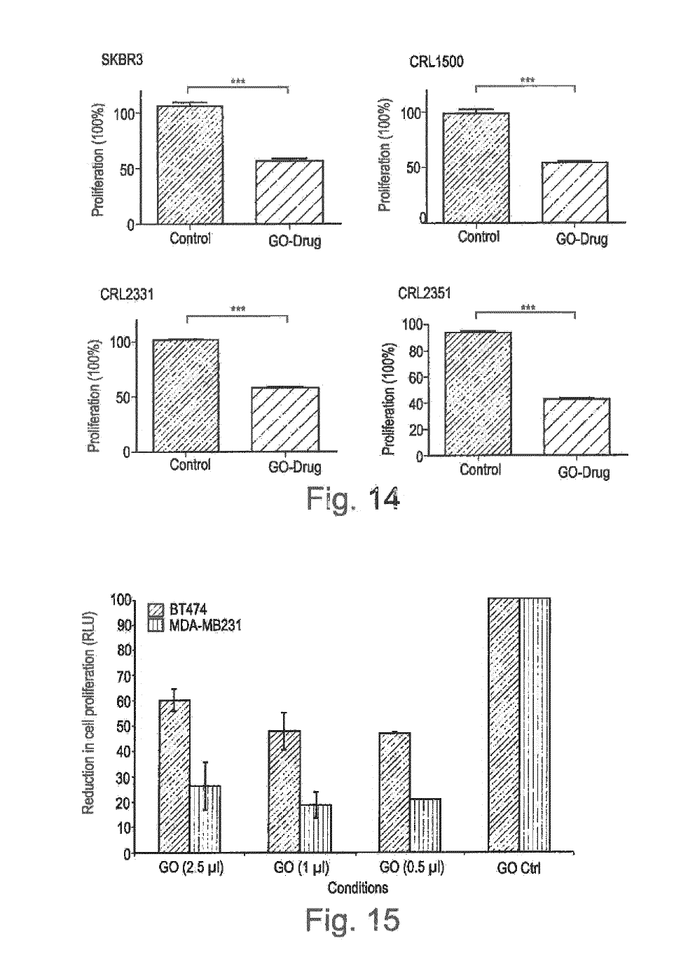

Such structure is for example illustrated in FIG. 1.

A support material in the form of platelets of flakes can in particular be independently, at least partially overlaid by the multilayered structure on its both sides, in particular when the support material is graphene oxide.

A support material under this form can in particular be present in nano-sized drug supports of the invention having a spherical or ellipsoidal architecture in the form of a core/shell type nanoparticle, as illustrated for example in FIG. 2.

According to a preferred embodiment, a support material of the invention is formed of gold nanoparticles functionalized with at least one carboxylate-containing radical.

According to a preferred embodiment, a support material of the invention is selected from a graphene-based or graphene derivative-based support material and gold nanoparticles functionalized with at least one carboxylate-containing radical, in particular from graphene, graphene oxide, an aminated-graphene, an aminated graphene oxide and gold nanoparticles functionalized with at least one carboxylate-containing radical.

In another embodiment, the support material of a nano-sized drug delivery structure according to the invention is formed, in whole or in parts, of nanometer-sized graphene, in particular of negatively or positively charged graphene quantum dots, more particularly of negatively charged graphene quantum dots, and especially of carboxylated graphene quantum dots.

According to this particular embodiment, the support material formed, in whole or in parts, of graphene quantum dots, preferably has a thickness (or diameter) comprised between 10 nm and 100 nm, preferably between 10 nm and 30 nm.

Multilayered Structure

A nano-sized drug support of the invention is further characterized in that it comprises a multilayered structure overlaying and/or surrounding at least partially the support material and comprises at least two layers of oppositely charged materials, i.e. at least one layer of positively charged material (a positively charged layer) and at least one layer of negatively charged material (a negatively charged layer).

The positively charged material(s) constituting the positive layer(s) of a multilayered structure of the invention can be chosen from polylysine, chitin, chitosan, chitosan derivatives, amidated polymers, polyarginine, polyhistidine and mixtures thereof.

A positively charged material of the invention is preferably polylysine. Accordingly, a positively charged layer of the invention preferably comprises at least polylysine, and in particular consists only of polylysine, more particularly polylysine with a molecular weight of between 1,000 and 50,000 Da, preferably between 1,000 and 5,000 Da.

A positively charged layer according to the invention preferably has a thickness comprised between 1 nm and 10 nm.

A negatively charged material of the invention is preferably selected from the group consisting of biopolymers; forms of carbon functionalized with at least one carboxylate-containing radical; and mixtures thereof.

Biopolymers according to the invention can in particular be selected from sulphated polysaccharides and more particularly selected from heparin, heparan sulphate, chrondroitin sulphate, dermatan sulphate, keratan sulphate, hyaluronic acid and polyglutamic acid. A biopolymer of the invention is preferably heparin.

The sulphated polysaccharide used as negatively charged material according to the invention may further contain a carboxyl group. Without being limited to a specific theory, the presence of a carboxyl group is advantageous for the activation and following conjugation of the sulphated polysaccharide with a drug active containing a sulfhydryl group.

A form of carbon functionalized with at least one carboxylate-containing radical is preferably a graphene oxide functionalized with at least one carboxylate-containing radical.

Accordingly, a negatively charged layer of the invention preferably comprises heparin, graphene oxide functionalized with at least one carboxylate-containing radical or mixtures thereof as negatively charged material(s), and in particular consists only of heparin, graphene oxide functionalized with at least one carboxylate-containing radical or of mixtures thereof as negatively charged material(s).

In a preferred embodiment, a negatively charged layer of the invention comprises heparin or graphene oxide functionalized with at least one carboxylate-containing radical as negatively charged material(s), and is preferably heparin.

According to a preferred embodiment, a multilayered structure of the invention comprises one or more layers of heparin as negatively charged material.

A negatively charged layer according to the invention preferably has a thickness comprised between 1 nm and 10 nm.

Preferably, a multilayered structure of the invention comprises at least one layer of polylysine as positively charged material and at least one layer of heparin as negatively charged material.

In a particular embodiment, a multilayered structure of the invention comprises at least one inner layer of heparin onto which the drug active is immobilized, in particular conjugated. The immobilization, in particular the conjugation, preferably involves a disulphide bond.

According to this embodiment, the multilayered structure of the drug delivery structure according to the invention preferably comprises at least one inner layer of heparin onto which the at least one drug active is conjugated by means of a disulphide bond.

A multilayered structure of the invention has in particular between 2 and 10 layers, more particularly between 2 and 7 layers.

A multilayered structure of the invention preferably has 2 layers, 3 layers, 4 layers, 5 layers, 6 layers, 7 layers, 8 layers, 9 layers or 10 layers.

In a preferred embodiment, the multilayered structure of the invention has at least 4 layers, more preferably at least 5 layers.

A multilayered structure of the invention more preferably has 3 layers, 5 layers, 7 layers or 9 layers. Even more preferably, a multilayered structure of the invention has 5 layers, 7 layers or 9 layers.

A multilayered structure of the invention is formed by alternating positively charged layers with negatively charged layers.

In the present invention, if the support material of the invention is positively charged, then a negatively charged material will be firstly overlaid, leading to a negatively charged first layer of the multilayered structure of the invention, being followed by a positively-charged second layer.

Alternatively, if the support material of the invention is negatively charged, then a positively charged material will be firstly overlaid, leading to a positively charged first layer of the multilayered structure of the invention, being followed by a negatively-charged second layer.

For example, when the support material of the invention is functionalized with at least one amine radical, then a negatively charged material will be firstly overlaid, leading to a negatively charged first layer of the multilayered structure of the invention, being followed by a positively-charged second layer.

According to an embodiment, the first layer of a multilayered structure of the invention is a positively charged layer, preferably a polylysine layer.

According to another embodiment, the first layer of a multilayered structure of the invention is a negatively charged layer, preferably heparin. An aminated graphene oxide can also be considered.

In a preferred embodiment, the multilayered structure is formed by alternating:

(i) layers of positively charged polylysine, with

(ii) layers of negatively charged heparin.

More particularly, the multilayered structure of a nano-sized drug delivery structure according to the invention consists in alternating layers of positively charged polylysine and layers of negatively charged heparin.

In a preferred embodiment, the multilayered structure of the invention comprises, or even consists, of:
a first layer of positively charged polylysine;
a second layer of negatively charged heparin onto which at least one drug active is immobilized;
a third layer of positively charged polylysine;
a fourth layer of negatively charged heparin onto which at least one drug active is immobilized; and
a fifth layer of positively charged polylysine.

A multilayered structure according to this embodiment can advantageously further comprise:
an additional sixth layer of negatively charged heparin, optionally onto which the at least one drug active is immobilized; and
an additional seventh layer of positively charged polylysine.

A multilayered structure according to this embodiment can optionally further comprise:
an additional eighth layer of negatively charged heparin, optionally onto which the at least one drug active is immobilized; and
an additional ninth layer of positively charged polylysine.

The first layer of a multilayered structure of the invention can overlay or surround partially or completely the support material onto which it is applied.

Preferably, the first layer completely overlays or surrounds the support material.

In particular, when the support material is in the form of a platelet or flake, the first layer overlays, partially or completely, preferably completely, the surface of the support material.

When only one face of the support material in the form of a platelet or flake is overlaid with a multilayered structure of the invention, the nano-sized drug support can be called a carpet, or nano-sized drug carpet.

In a particular embodiment, the two faces of the support material in the form of a platelet or flake are overlaid with a multilayered structure of the invention. Preferably, the first layer of the multilayered structure on both faces overlays completely the support material.

In a particular embodiment, the support material as defined previously is surrounded, partially or completely, preferably completely, by the first layer of the multilayered structure of the invention.

The layers of the multilayered structure of the invention different from the first layer can overlay or surround partially or completely, independently, the layer just beneath them.

The total thickness (i.e. diameter for a sphere) of the multilayered structure on either side of the support is preferably inferior or equal to 250 nm.

Drug Active

A nano-sized drug delivery structure of the invention comprises at least one drug active agent immobilized onto at least one layer, preferably onto at least one inner layer, of the multilayered structure of the invention.

The expressions “drug agent”, “drug active agent” and “drug active” are similar expressions used in the present text in order to designate the exact same thing.

In a preferred embodiment, a drug active of the invention is at least immobilized onto a negatively charged material of a multilayered structure of the invention, in particular onto a negatively charged layer of a multilayered structure of the invention.

Said immobilization of the drug active agent can involve a disulphide bound or a free amine moiety, preferably a disulphide bound. What is important is that the moiety implicated in the immobilization is not crucial for the activity of structure of the drug active agent.

The immobilization of the drug active agent can occur through a covalent bound, electrostatic interactions, hydrogen binding and/or Van der Waals forces.

A drug active according to the invention can be selected from the group consisting of peptides, proteins, small chemical compounds, and mixtures thereof.

A drug active according to the invention is preferably selected from peptides, preferably from cationic peptides, and more particularly from cationic lytic peptides.

Independently, a drug active of the invention can be selected from the group consisting of anticancer agents, antibacterial agents, antiviral agents, antifungal agents, analgesics, antihyperlipidemic agents, antidepressants, β-receptor blockers, calcium channel blockers, diuretics, cardiac glycosides, antiarrhythmics, nitrate, antianginals, vasoconstrictors, vasodilators, antihypertensive agents, agents affecting the central nervous system, agents for musculoskeletal disorders, antiallergy agents, mast cell inhibitors agents, anti-inflammatory agents, skin agents, eye disorders agents, obstetrics agents and gynecologic agents, and mixtures thereof, and is preferably at least one anticancer agent.

According to a preferred embodiment, the drug active agent is conjugated by means of a disulphide bond to one or more negatively charged material(s) of one or more inner layer(s) of a multilayered structure of a delivery structure according to the invention.

In this respect, the drug active may originally contain a sulfhydryl group or may have been modified in order to contain a sulfhydryl group.

In a particular embodiment, a drug delivery structure of the invention comprises different types of drug actives.

When different drug actives are immobilized in a nano-sized drug delivery structure of the invention, the different drug actives can be immobilized onto different layers or onto the same layer(s).

For example, if a nano-sized drug delivery structure of the invention comprises 2 different drug actives, one of the drug active can be immobilized onto a given layer, in particular onto an inner layer of the multilayered structure of the invention, while the other drug active is immobilized onto another layer, in particular onto another inner layer of the multilayered structure of the invention.

In another example, if a nano-sized drug delivery structure of the invention comprises 2 different drug actives, the two drug actives can be immobilized onto the same layer, in particular onto the same inner layer of the multilayered structure of the invention, or onto the same layers, in particular onto the same inner layers of the multilayered structure of the invention.

In a particular embodiment, a drug delivery structure of the invention comprises only one type of drug actives.

Preferably, the content of drug active in a drug delivery structure of the invention is comprised between 1 and 120 picomol/nm², preferably between 50 and 100 picomol/nm².

In a particular embodiment, the drug active of the invention can be labelled with at least one detectable molecule or substance, in particular selected from the group consisting of enzymes, prosthetic groups, fluorescent materials, luminescent materials, bioluminescent materials and radioactive materials.

A drug active of the invention can for example be labeled with a fluorescein isothiocyanate (FITC) fluorophore or to the red fluorescent dye tetramethylrhodamine (TMR).

Processes for labelling a given drug active are well known to the man skilled in the art.

A drug active of the invention can be immobilized, in particular conjugated, onto a layer of the invention through different methods. For example, the drug active can be immobilized on heparin through a process consisting in forming PDPH-modified heparin in formamide in the presence of EDC/NHS, the PDPH-modified heparin being then conjugated with a SH-containing drug via disulphide exchange reactions.

Nano-Sized Drug Delivery Structure Preparation and Uses A nano-sized drug delivery structure of the invention can be obtained according to different methods, such as the one illustrated in the examples as well as in FIG. 3.

Accordingly, the present invention relates to a process for preparing a nano-sized drug delivery structure of the invention wherein the layers of the multilayered structure are formed by chemical deposition of each material in the appropriate order on the support material.

The deposition of multilayers on a support can be carried out in an aqueous medium supplemented with a low-density non-ionic surfactant such as poloxamers, such as Pluronic® F68, in order to reduce the aggregation of the particles during isolation processes.

Suitable poloxamers that can be used in a process according to the invention are the ones marketed under the names Pluronic® 25R4, Pluronic® 31R1, Pluronic® F68, Pluronic® F77, Pluronic® F87, Pluronic® F88, Pluronic® FTL61, Pluronic® L10, Pluronic® L35, Pluronic® L62LF, Pluronic® N-3, Pluronic® L92, Pluronic® P103, Pluronic® P104, Pluronic® P105, Pluronic® P123, Pluronic® P65, Pluronic® P84 or Pluronic® P85, by the company Sigma Aldrich.

The said low-density non-ionic surfactant preferably has a density of less than 1.1 g/cm³, preferably ranging from 1 to 1.08 g/cm³, more preferably ranging from 1.01 to 1.06 g/cm³.

Such a low-density non-ionic surfactant can be present in the aqueous medium so as to represent about 10% by weight of the total weight of the medium.

In particular, the deposition steps can be interspersed by washing and/or centrifugation and/or ultracentrifugation steps, preferably by washing and centrifugation steps.

In a particular embodiment, the nano-sized structure of the invention is labelled with at least one detectable molecule or substance, in particular selected from the group consisting of enzymes, prosthetic groups, fluorescent materials, luminescent materials, bioluminescent materials and radioactive materials.

Said detectable molecule or substance can be immobilized on any layer of the multilayered structure of the nano-sized structure of the invention and is preferably immobilized on the outer layer of the multilayered structure of the nano-sized structure of the invention.

Said detectable molecule or substance is in particular immobilized in a way that it does not alter the delayed and controlled diffusion of the at least one drug active present in the nano-sized drug delivery structure.

In a preferred embodiment, the nano-sized structure of the invention comprises at least one targeting agent immobilized on the external surface of the outer layer of the multilayered structure of the nano-sized structure of the invention.

This targeting agent can in particular be selected from the group consisting of an antibody, an antibody fragment, an oligonucleotide, a peptide, a hormone, a ligand, a cytokine, a peptidomimetic, a protein, a carbohydrate, a chemically modified protein, a chemically modified nucleic acid, a chemically modified carbohydrate that targets a known cell-surface protein, and an aptamer.

This targeting agent is preferably an antibody or an antibody fragment, in particular an antibody or an antibody fragment that targets cancer cells.

Antibodies appropriate for the present invention also include antibody fragments or modified products thereof, provided that they can be suitably used in the present invention.

Appropriate antibody fragments comprise at least one variable domain of an immunoglobulin, such as single variable domains Fv, scFv, Fab, (Fab′)² and other proteolytic fragments.

The terms “antibody” and “antibodies” further include in the present invention chimeric antibodies; human and humanized antibodies; recombinant and engineered antibodies, conjugated antibodies, and fragments thereof.

Humanized antibodies are antibodies wherein the complementarity determining regions (CDRs) of an antibody from a mammal other than human (e.g., a mouse antibody) are transferred into the CDRs of human antibodies.

Chimeric and humanized antibodies can be made according to standard protocols such as those disclosed in U.S. Pat. No. 5,565,332. Other antibody formats are described in, for example, “Antibody Engineering”, McCafferty of a/. (Eds.) (IRL Press 1996).

The antibodies of the present invention may be polyclonal or monoclonal antibodies. Preferably, the antibodies are monoclonal. Methods of producing polyclonal and monoclonal antibodies are known in the art and described generally, e.g., in U.S. Pat. No. 6,824,780.

The source of the antibodies described herein is not particularly restricted in the present invention; however, the antibodies are preferably derived from mammals, and more preferably derived from humans.

Preferably, the antibodies or fragments of antibodies used in the present invention target cancer cells.

The targeting agent may further be derived from a molecule known to bind to a cell-surface receptor. For example, the targeting moiety may be derived from low density lipoproteins, transferrin, EGF, insulin, PDGF, fibrinolytic enzymes, anti-HER2, annexins, interleukins, interferons, erythropoietins, or colony-stimulating factor.

The targeting agent of the nano-sized structure of the invention can target any specific tissue, organ or epitope of interest in the organism of an individual to which said nano-sized structure is administered.

Preferably, the targeting agent's targets are in accordance with the drug active immobilized on the inner layer(s) of the multilayered structure of the nano-sized drug delivery structure. For example, if the drug active is an anticancer agent, the targeting agent is chosen so as to target cancer cells.

Furthermore, a nano-sized drug delivery structure of the invention can be used for the manufacture of a pharmaceutical composition and/or of a medicament.

Accordingly, the present invention also relates to a pharmaceutical composition comprising, in a pharmaceutically acceptable carrier, at least one nano-sized drug delivery structure of the invention.

The present invention also relates to a medicament comprising at least a nano-sized drug delivery structure of the invention.

A pharmaceutical composition and/or of a medicament can comprise nano-sized drug delivery structures of the invention in a content ranging from 1% to 30% by weight, in particular from 5% to 10% by weight, relative to the total weight of the composition or medicament.

Preferably, the drug active present in a nano-sized drug delivery structure of the invention used for the manufacture of a pharmaceutical composition and/or of a medicament is an anti-cancer agent, said pharmaceutical composition and/or medicament being intended to be used in cancer therapy.

After preparation of a drug delivery structure according to the invention, and/or of a pharmaceutical composition or medicament of the invention comprising at least one drug delivery structure of the invention, the drug delivery structure, pharmaceutical composition or medicament is preferably lyophilized. Once lyophilized, said delivery structure, composition or medicament can stored for long periods of time.

Lyophilization may be performed via any method known in the field.

A pharmaceutical composition or medicament according to the invention is preferably reconstituted in a saline suspension before administration.

When the pharmaceutical composition or medicament is reconstituted in a saline suspension before administration, said reconstitution steps preferably takes place less than 2 hours, in particular less than 1 hour, more particularly less than 30 minutes before administration.

In particular, a pharmaceutical composition of medicament of the invention is preferably administered as a liquid.

The present invention is illustrated by the following examples, given purely for illustrative purposes, with reference to the following figures.

### EXAMPLES

### Example 1

Therapeutic Agent/Drug Active Agent

The drug agent chosen for the study is a cationic peptide drug, in particular an anti-cancer agent previously characterized as being a cationic lytic peptide, whose mechanism of action is based on disintegrating the cell membrane, leading to cell death.

This peptide has the following sequence SEQ ID NO: 1:

(SEQ ID NO: 1)
KLLLKLLKKLLKLLKKK

Incubation of MDA-MB-231 (human breast basal epithelial cancer cells) with different concentrations of the peptide has been reported to show a dose-dependent reduction in cell proliferation (Kawamoto et al., Mol. Cancer Ther., 2013. 12(4): p. 384-393).

The peptide drug was chemically synthesized with the addition of a fluorescein isothiocyanate (FITC) fluorophore at the N-terminal of the peptide and with an additional cysteine at the C-terminal. This enabled the monitoring of the presence of the peptide using green fluorescence. The concentration of the peptide was proportional to the fluorescence intensity and calibration curves can be derived to measure peptide drug concentration.

Coupling of the Therapeutic Agent to Heparin

The fluorescent therapeutic peptide is covalently linked to heparin via the addition of a chemical crosslinker, PDPH ((3-(2-pyridyldithio)propionyl hydrazide)).

In particular, heparin is reacted with EDC/NHS (carbodiimide/N-Hydroxysuccinimide ester) and PDPH in formamide as buffer.

After buffer change using dialysis and freeze-drying, lyophilized sulfhydryl-derived heparin is obtained. Displacement Reactions at the disulphide Linkage is used to conjugate cysteine containing peptide to sulfhydryl-heparin.

Example Procedure for Fabrication of a Multilayered Drug Carpet

The procedure for the fabrication of the multilayered drug carpet is summarized in the schematic in FIG. 3.

Graphene oxide nanoflakes were used as support material and were initially characterized using transmission electron microscopy as indicated here-above. This showed the presence of uniformly distributed 2D islands with typical lateral diameter between 50 to 200 nm.

6 mg of nanosized graphene oxide flakes are centrifuged for 10 to 20 mins at 14,000 to 50,000 xg and sonicated using a low-power ultrasonicator bath.

Addition of polylysine (1-4 mg/ml) in 100-1000 ul of volume is followed by incubation for 1 h at 4° C. on a rotating mixer.

Addition steps of heparin incubation are interspersed by washing (with 1×PBS) and centrifugation steps for 10-20 mins at 14,000 to 50,000 xg.

Following steps include addition of FITC-fluorescent peptide and incubation for 5 minutes to 1 hour at 4° C. or room temperature, using 1,000 μl of 1 mg/ml of peptide. The buffer can be 50%:50% 1×PBS and DMSO. Repetitive steps of incubation, centrifugation and washing steps are carried out for every layer added.

In order to study the structural characteristics and topology of the nano-sized drug delivery structure, each fabrication step was characterized using atomic force microscopy (AFM).

The dimensions of the drug delivery structures were measured for their parameters in the x, y and z dimensions. This was carried out for each step which includes, in the present case (i) graphene oxide alone, (ii) graphene oxide plus polylysine, (iii) graphene oxide plus polylysine plus the heparin conjugated drug, then each of the following additional layered step.

The experiments showed that the graphene oxide was approximately 1 nm (FIG. 1). The polylysine layer was measured to be approximately 3 nm (3.2 nm) in thickness and the heparin-drug layer was measured to be approximately 4 nm (4.1 nm) in thickness.

The experimental data from the AFM experiments also depict a pyramidal type structure, which allows us to define the metamaterial structure as a drug carpet. The platelets of graphene oxide can be either individually separated or partially or completely aggregate together.

Release of the Therapeutic Peptide Upon Reduction of the Disulphide Bond

The release of the therapeutic peptide following reduction of the disulphide bond is achieved using the addition of a reducing agent DTT (Dithiothreitol), and heating to 56° C. for 1 hour. The same results have also been obtained by heating to physiological temperatures (37° C.) for 1 hr.

Using MALDI-TOF mass spectroscopy, the results presented in FIG. 4 have been obtained.

Releasing of the peptide by cleavage of the disulphide bond is well observed.

Indeed, MALDI spectroscopy analysis shows the release of therapeutic anticancer drug upon hydrolysis of disulphide bond enabled in reducing conditions.

Different conditions have been compared and are represented in FIG. 4.

Firstly, the peptide (5 μL) only linked to FITC via an aminohexanoic acid (Ahx) spacer in the presence of H₂O (5 μL) (FITC-Ahx-Peptide+H₂O—1 h 56° C.). The presence of the FITC-Ahx-Peptide is clearly noticeable.

Secondly, the peptide (5 μL) only linked to FITC via an aminohexanoic acid (Ahx) spacer in the presence of DTT (5 μL-1M) (FITC-Ahx-Peptide+DTT—1 h 56° C.). The presence of the FITC-Ahx-Peptide is clearly noticeable and is similar to what is observed in the first condition with H₂O instead of DTT.

The third and fourth conditions are when the peptide (5 μL) is both linked to heparin as indicated here-above and to FITC via an aminohexanoic acid (Ahx) spacer in a drug delivery structure of the invention.

In the third condition, this structure is observed in the presence of DTT (5 μL-1M). The releasing of the marked peptide is well observed, as it can be seen in FIG. 4.

On the contrary, in the fourth condition where the drug delivery structure is observed in presence of only H₂O (5 μL), only few peptides are released compared to the other conditions.

In cellular conditions, tumour cells are known to have a more reducing environment than normal healthy cells due to higher levels of GSH, reducing conditions that were simulated in the present experiments by the addition of DTT.

Measuring the Diffusion Properties of the Drug Delivery Structure

In order to measure the rate of diffusion of the therapeutic drug, the fluorescent tag linked to the peptide was monitored. In particular, the rate of diffusion of the therapeutic drug was observed at each step of the drug delivery structure construction as indicated here-above.

FIG. 5 represents a schematic representation of the method used for the first condition when Graphene oxide as support material was used.

Schematic representation of graphene oxide deposited onto PET membrane (a), fluorescent peptide with anti-proliferative activity added to Transwell cell culture insert (PET membrane) at the beginning of the experiment (t=0) (b) and at each time point during the experiment, which measures the diffusion of the fluorescent peptide concentration in the lower culture well chamber.

The same method has been used to measure the rate of diffusion at each step of the drug delivery structure construction and without any step of the drug delivery structure (peptide alone).

Graphene oxide dispersion in water was prepared at the appropriate concentration and dried onto translucent cell culture insert made from PET membrane, with a porosity of 0.4 microns.

Fluorescent anti-proliferative peptide as described here-above was added into the insert and the fluorescence intensity was read from the bottom of the wells plate. Calibration curves of peptide concentration vs. fluorescence intensity showing a linear positive regression relationship (r=0.8) were used to determine the peptide concentration.

The results obtained are represented in FIG. 6A.

Measuring the rate of diffusion of each type of layered structure shows that the 5-layered and the 7-layered drug metamaterial (nano-sized drug delivery structure) of the invention is capable of retarding the rate of diffusion of the therapeutic drug by 50% compared to the free drug delivery structure.

This shows that the 5-layered and the 7-layered metamaterial drug delivery structures are capable of significantly reducing the rate of diffusion of the therapeutic drug molecule.

Following the Internalization of a Graphene Oxide Drug Metamaterial of the Invention

Video microscopy is used to follow the internalization of the graphene oxide drug metamaterial describe here-above using transmission microscopy and fluorescence microscopy.

The cells were plated on 18 mm glass coverslips, placed in a Ludin Chamber (Life Imaging Services), the nanoparticle structures were added in cell media at different dilutions.

Cellular internalization was followed under an inverted microscope (Zeiss AxioObserver Z1) equipped with an oil immersion objective (63×aPL APO HCX, 1.46 NA), a temperature control system (TempControl S, Zeiss) and CO₂ control (CO₂ Control S, Zeiss), a piezo translator for Z-stack acquisition and a EMCCD camera (EVOLVE 512 Photometrics).

Image acquisition and analysis were performed using Metamorph Software (Universal Imaging).

A Z-series of fluorescence images were recorded every 2 min for 2 hours followed by 20 min for 12 hours at 1 μm increment without a binning additionally transmission microscopy without z series. Z stack maximum image projection was obtained using Metamorph Software for fluorescent images only.

### Example 2

Therapeutic Agent/Drug Active Agent

The first drug agent chosen for this study is the cationic peptide drug of sequence SEQ ID NO:1 defined in example 1.

The second drug agent used in this study is a small molecule drug, known as a mitotic inhibitor, called Mertansine.

The peptide drug was chemically synthesized with the conjugation of tetramethylrhodamine (TMR) to the N-terminal of the peptide and with an additional cysteine at the C-terminal. This enabled the monitoring of the presence of the peptide using green fluorescence. The concentration of the peptide was proportional to the fluorescence intensity and calibration curves can be derived to measure peptide drug concentration.

Coupling of the Therapeutic Agent to Heparin

Firstly, high molecular weight heparin is functionalized using a crosslinker PDPH, to add a —SH moiety onto the —COOH of the heparin chain.

Then, the coupling process is similar to the one described in Example 1.

FIG. 8 shows the synthesis of heparin-mertansine from heparin-PDPH by displacement reaction at the disulphide linkage.

Example Procedure for Fabrication of a Multilayered Drug Carpet

The procedure for the fabrication of the multilayered drug carpet is similar to the one of example 1. In the present example, the chemical deposition of polylysine and heparin layers is performed in presence of a poloxamer surfactant, marketed under the trade name Pluronic F68® by the company Sigma Aldrich.

Graphene oxide nano flakes were used as support material and were initially characterized using transmission electron microscopy as indicated here-above. This showed the presence of irregular shard-like shaped individual flakes with the largest diameter being on average 90 to 200 nm.

After spin-coating a solution of the metamaterial onto freshly cleaved Mica discs, the resulting surface morphology is examined directly using high resolution AFM imaging, in order to study the structural characteristics and topology of the nano-sized drug delivery structure.

The dimensions of the drug delivery structures were measured for their parameters in the x, y and z dimensions. This was carried out for each step which includes, in the present case (i) graphene oxide alone, (ii) graphene oxide plus polylysine, (iii) graphene oxide plus polylysine plus the heparin conjugated drug, then each of the following additional layered step.

The experiments showed that the graphene oxide was approximately 1 nm height (0.8 nm) (FIG. 1). The polylysine layer was measured to be approximately 3 nm (3.2 nm) in thickness and the heparin-drug layer was measured to be approximately 4 nm (4.1 nm) in thickness. The last layer has approximately 3 nm height (3.4 nm) confirming that the alternating layers of polylysine and heparin are conserved during substrate synthesis.

The experimental data from the AFM experiments also depict a pyramidal type structure, which allows us to define the metamaterial structure as a drug carpet.

Furthermore, fluorescent peptide drug was used in order to determine the percentage of peptide drug disposition, which was estimated to be 1 percent.

Release of the Therapeutic Peptide Upon Reduction of the Disulphide Bond

The release of the therapeutic peptide following reduction of the disulphide bond is achieved using the addition of a reducing agent DTT (Dithiothreitol), and heating to 56° C. for 1 hour. The same results have also been obtained by heating to physiological temperatures (37° C.) for 1 hr.

Using MALDI-TOF mass spectroscopy, the same results as the one presented in FIG. 4 have been obtained.

Releasing of the peptide by cleavage of the disulphide bond is therefore well observed.

Measuring the Diffusion Properties of the Drug Delivery Structure

In order to measure the rate of diffusion of the therapeutic drug, the fluorescent tag linked to the peptide was monitored. In particular, the rate of diffusion of the therapeutic drug was observed at each step of the drug delivery structure construction as indicated here-above.

The measuring method used for the first condition when Graphene oxide as support material was used is the same as in example 1 (FIG. 5).

The results obtained are represented in FIG. 9.

Measuring the rate of diffusion of each type of layered structure shows that the 5-layered and the 9-layered nano-sized drug delivery structure of the invention is capable of drastically retarding the rate of diffusion of the therapeutic drug compared to the free peptide.

This shows that the 5-layered and the 9-layered metamaterial drug delivery structures are capable of significantly reducing the rate of diffusion of the therapeutic drug molecule.

Following the Internalization of a Graphene Oxide Drug Metamaterial of the Invention

Immunofluorescence analysis of breast cancer cells, T47D, was carried out to follow the internalization of the graphene oxide drug metamaterial described here-above.

The protocol used to follow the internalization of the graphene oxide drug metamaterial is the same as the one described in example 1. Before the nanoparticles are added to cells, the nanoparticle solution is centrifuged at high speed (14K rpm for 10 min) and the solution changed to the appropriate cell culture media.

The results are the same as the ones of example 1 and show that the cells are capable of internalizing the peptide after 20 minutes.

The uptake mechanism of the GO nanodrug structures was then investigated using various endocytosis inhibitors in comparison with the graphene oxide drug metamaterial described here-above, free of any drug active (GO control).

Different endocytosis inhibitors were tested:
Cytochalasin D, an inhibitor of macropinocytosis-dependent endocytosis in a dosage of 10, 5 and 1 (μM);
Genistein, an inhibitor of caveolae-mediated endocytosis in a dosage of 100, 30 and 25 (μM); and
Chlorpromazine (CMZ), an inhibitor of clathrin-mediated endocytosisin a dosage of 5, 1 and 0.5 (μM).

Cellular internalization of each nanostructure was followed as described here-after.

SKBR3 cells were seeded to a density of 1.0×10⁵ cells per well in a 12-well plates followed by an overnight incubation. The cells were rinsed with PBS and pre-incubated with different concentrations of the appropriate endocytic inhibitor in serum-free RPMI 1640 media, for 1 hour. The media was then replaced with the same concentration of inhibitors in serum-containing RPMI 1640 media, the nanoparticles were added and cells were incubated for 15 mins. Following incubation, the medium was removed and cells were rinsed several times with cold PBS. The cells were collected with trypsin EDTA and analyzed using flow cytometry analysis.

Cytochalasin D significantly inhibited the internalization of GO nanodrug structures in a dose-dependent manner. On the other hands, Genistein and Chlorpromazine (CMZ) did not show any notable inhibition on the cellular uptake (FIG. 10).

Evaluating the Targeted Therapy Ability of a Nano-Sized Drug Delivery Structure According to the Invention

The GO-drug nanostructures has been modified to provide targeted therapy by the addition of an antibody. A HER2 antibody has been used to potentially target HER2 overexpressing cancers, which make up 20 percent of all breast cancer subtypes.

The specificity of the GO-HER2 antibody nanodrug structures was seen by preincubating breast cancer cells which overexpress HER2, SKBR3 human breast basal epithelial cancer cells and BT474 cells, or express lower levels of HER2, MDAMB231 and MCF7.

The specificity of the of GO-HER2 antibody nanodrug structures was seen by preincubating breast cancer cells which overexpress HER2, SKBR3 cells, with Lapatinib. Lapatinib is a known HER2 tyrosine kinase inhibitor, which induces stabilization and accumulation of HER2.

The results showed that there was a successful dose-dependent reduction in intake, and therefore a reduction of uptake efficiency of GO-HER2 antibody nanodrug structures upon addition of Lapatinib (FIG. 11). In addition, GO-HER2 antibody nanodrug structures were only able to target cells which express high quantities of HER2 due to gene amplification.

Using transmission electron microscopy, the internalization of the GO-HER2 antibody nanodrug structures has been analyzed. Mertansine was localized using FITC-labelled polylysine.

As shown in FIGS. 12 A and B, the particles are located on the cell surface, in the cytoplasm and in lysosomes. Localization of the GO-HER2 nanoparticles to the lysosomal cell compartments were further confirmed by colocalization with the lysosomal marker, LAMP1.

Comparable results were achieved for both the fluorescent peptide drug and mertansine.

Determining the Efficient Activity and Specific Targeting of the Drug Nanostructures

In order to determine the therapeutic effect of the nano-sized drug delivery structures, in vitro cell proliferation tests were carried out.

The anti-cancer agents used in this study is the peptide of sequence SEQ ID NO: 1 defined here-above and mertansine.

The 5-layered and the 9-layered nano-sized drug delivery structures prepared here-above was compared in terms of the rate of cell proliferation following 24 hours and 48 hours of incubation.

There was a retardation on release of the lytic peptide and so a delay in the decrease in cell proliferation compared to the GO control conditions (FIGS. 13 A and B (in which GO-Drug refers to the 9-layered GO)).

Release of mertansine drug from GO-HER2 nanodrug structures were even more effective in reduction of cell proliferation and active in four HER2-overexpressing cell breast cancer cell lines (FIG. 14, in which GO-Drug refers to the 9-layered GO).

Specificity was further shown by better activity in the HER2 overexpressing cell line BT474 compared to lower HER2 expressing cell line, MDMB231.

Using the drug deposition estimation and reduction in cell proliferation, it has been calculated that 75 pg of mertansine is deposited onto GO nanostructures which is able to achieve 95 percent of cytotoxity in HER2 overexpressing cells.

The sustained diffusion of drug delivery from the GO-HER2 nanodrug structures was analyzed by measuring the drug penetration in the interior of tumour spheroids.

This experiment is based on the general trend for the variation of drug concentration in a tumour. Indeed, the drug concentration in a tumour increases with time to reach a maximum value, then the concentration of the drug decreases gradually until reaching a stable value.

A drug penetration experiment using 3D cultured tumor spheroids was performed. The results unambiguously showed that GO-HER2 nanodrug structures were more effective in penetrating deeper into the interior of the tumor spheroids with respect to free peptide drug (FIG. 15, in which GO refers to the 9-layered GO). Therefore, the GO-HER2 nanodrug structures are efficient in achieving sustained drug diffusion compared to free peptide drug.

It was also shown that a drug structure containing further layers achieved better tumour penetration. This proves that that there is sustained drug delivery.

1. A nano-sized drug delivery structure for delayed and controlled diffusion of at least one drug active, said drug delivery structure comprising at least:
one support material selected from the group consisting of:
a metal support material functionalized with at least one carboxylate-containing radical or at least one amine function,
a graphene-based or graphene derivative-based support material, and
a calcium carbonate or calcium phosphate nanoparticle support material;
one multilayered structure overlaying and/or surrounding at least partially said support material and comprising at least two layers of oppositely charged materials:
the positively charged material(s) being chosen from polylysine, chitin, chitosan, chitosan derivatives, amidated polymers, polyarginine, polyhistidine and mixtures thereof;
the negatively charged material(s) being selected from the group consisting of sulphated polysaccharides; and
one drug active agent conjugated by means of a disulphide bond to one or more negatively charged material(s) of one or more inner layer(s) of the said multilayered structure.

2. The drug delivery structure according to claim 1,
wherein the support material is a graphene-based or a graphene derivative-based support material.

3. The drug delivery structure according to claim 1 wherein said multilayered structure comprises at least one layer of polylysine as positively charged material and at least one layer of heparin as negatively charged material.

4. The drug delivery structure according to claim 1 wherein the drug active is selected from the group consisting of anticancer agents, antibacterial agents, antiviral agents, antifungal agents, analgesics, antihyperlipidemic agents, antidepressants, β-receptor blockers, calcium channel blockers, diuretics, cardiac glycosides, antiarrhythmics, nitrate, antianginals, vasoconstrictors, vasodilators, antihypertensive agents, agents affecting the central nervous system, agents for musculoskeletal disorders, antiallergy agents, mast cell inhibitors agents, anti-inflammatory agents, skin agents, eye disorders agents, obstetrics agents and gynecologic agents, and mixtures thereof,
wherein said drug active originally contains a sulfhydryl group or has been modified in order to contain a sulfhydryl group.

5. The drug delivery structure according to claim 1 wherein the multilayered structure comprises at least one inner layer of heparin onto which the at least one drug active is conjugated by means of a disulphide bond.

6. The drug delivery structure according to claim 1 wherein the support material is in the form of platelets or flakes.

7. The drug delivery structure according to claim 20, wherein the support material has a thickness of between 0.1 nm and 5 nm.

8. The drug delivery structure according to claim 1, wherein said drug delivery structure has a spherical or ellipsoidal architecture in the form of a core/shell type nanoparticle comprising a core formed in all or part of the support material and a shell formed of the multilayered structure surrounding at least partially the core.

9. The drug delivery structure according to claim 1, wherein said multilayered structure consists in alternating layers of positively charged polylysine and layers of negatively charged heparin.

10. The drug delivery structure according to claim 1, wherein the multilayered structure is a 5-layered structure, a 7-layered structure or a 9-layered structure.

11. The drug delivery structure according to claim 1, wherein a targeting agent is immobilized on the external surface of the outer layer of the multilayered structure.

12. A process for preparing a nano-sized drug delivery structure as claimed in claim 1, wherein the layers of the multilayered structure are formed by chemical deposition of each material in the appropriate order on the support material.

13. The process according to claim 12, wherein the layers of the multilayered structure are formed by chemical deposition in an aqueous medium supplemented with a non-ionic surfactant.

14. A method of treatment using a nano-sized drug delivery structure as claimed in claim 1 for the manufacture of a pharmaceutical composition.

15. A pharmaceutical composition comprising, in a pharmaceutically acceptable carrier, at least one nano-sized drug delivery structure according to claim 1.

16. The drug delivery structure according to claim 1 for delayed and controlled diffusion of said drug active agent in an aqueous medium.

17. The drug delivery structure according to claim 1, wherein the negatively charged material(s) is(are) selected from the group consisting of heparin, heparan sulphate, chrondroitin sulphate, dermatan sulphate, keratan sulphate, hyaluronic acid and polyglutamic acid.

18. The drug delivery structure according to claim 1, wherein the support material is graphene oxide.

19. The drug delivery structure according to claim 4, wherein the drug active is one anticancer agent.

20. The drug delivery structure according to claim 1, wherein the support material is a graphene oxide in the form of platelets or flakes.

21. The drug delivery structure according to claim 20 wherein the support material has a thickness between 0.5 nm and 2 nm.

22. The drug delivery structure according to claim 11, wherein a targeting agent is conjugated on the external surface of the outer layer of the multilayered structure.

23. The drug delivery structure according to claim 11, wherein the said outer layer is a polylysine layer.

24. The process of claim 12, wherein the deposition steps are interspersed by washing, centrifugation, and/or ultrafiltration steps.

25. The process of claim 13 wherein said non-ionic surfactant is a low-density non-ionic surfactant.

26. The process of claim 25 wherein the low-density non-ionic surfactant is chosen from poloxamers.

27. The method as set forth in claim 14 wherein said pharmaceutical composition is intended to be used in cancer therapy.

28. The method of claim 27 further comprising the step of administering the pharmaceutical composition to a subject in need thereof.


SEQUENCE LISTING (1 sequence)

SEQ ID NO: 1 - protein, 17 aa, Artificial Sequence
  anti-cancer cationic lytic peptide
  anti-cancer cationic lytic peptide
KLLLKLLKKLLKLLKKK